(19) United States
(12) Patent Application Publication
(10) Pub. No.: US 2025/0339174 A1
(43) Pub. Date: Nov. 6, 2025

(54) Needle-Guiding Systems and Methods for Establishing Vascular Access

(71) Applicant: Bard Access Systems, Inc., Salt Lake City, UT (US)

(72) Inventors: Tyler L. Durfee, Stansbury, UT (US); Shayne Messerly, Kaysville, UT (US); Shawn Ray Isaacson, Layton, UT (US)

(21) Appl. No.: 18/652,728

(22) Filed: May 1, 2024

(51) Int. Cl. A61B 17/34; A61B 90/00

(52) U.S. Cl. CPC A61B 17/3403; A61B 90/361; A61B 90/39; A61B 2017/3413; A61B 2090/365

(57) ABSTRACT

Needle-guiding systems and methods can facilitate establishing vascular access with a nonmagnetic needle. For example, a method of a needle-guiding system can include detecting and registering registration marks about a patient by patient-facing cameras of an alternative reality (“AR”) device, the AR device thereby establishing its location and orientation relative to a target area of the patient. The method can also include anchoring ultrasound images of the target area as viewed through a display screen of the AR device, the ultrasound images anchored about the patient relative to either an instant or previous location and orientation of the ultrasound probe; and providing an instant virtual needle trajectory of the needle as viewed through the display screen of the AR device, thereby indicating to a clinician whether the needle is properly oriented for establishing vascular access to a target vessel of the ultrasound images.

### BACKGROUND

Vascular access by way of, for example, placement of a catheter into a peripheral blood vessel provides an effective means for withdrawing blood, transfusing blood, delivering medications, or providing nutrition to a patient over a period of days, weeks, or even months. Such vascular access is initially established by a percutaneous puncture with a needle, and a variety of ultrasound systems having magnetic needle-guiding technology exist to facilitate first-stick success when establishing vascular access. While the foregoing ultrasound systems greatly improve first-stick success, a clinician has to divide his or her spatial attention between two different spatial regions when establishing vascular access. Indeed, the clinician has to divide his or her spatial attention between 1) a target area of a patient for the vascular access, where an ultrasound probe is used for ultrasound imaging, and 2) a display rendering corresponding ultrasound images of the target area. Having to divide spatial attention between the target area and the display can be difficult when simultaneously ultrasound imaging and establishing vascular access to a peripheral blood vessel with a needle. Notably, such difficulty can be pronounced with less experienced clinicians and older clinicians having reduced lens flexibility in their eyes. Further, the foregoing ultrasound systems also require the clinician to divide use of his or her hands between two different tasks, specifically, manipulating the ultrasound probe for the ultrasound imaging and manipulating the needle for establishing vascular access. What is needed are needle-guiding systems that allow clinicians to maintain their spatial attention in the target area while establishing vascular access, as well as provide the option of using both hands for establishing vascular access.

Disclosed herein are needle-guiding systems and methods that address the foregoing needs.

### SUMMARY

Disclosed herein is needle-guiding system for establishing vascular access. The needle-guiding system includes, in some embodiments, an ultrasound probe, a console, and a wearable alternative-reality (“AR”) device. The ultrasound probe includes an ultrasound sensor array disposed in a probe head of the ultrasound probe. The ultrasound sensor array is configured to emit source ultrasound signals into a patient and receive echoed ultrasound signals from the patient when holding or sliding the probe head over skin of the patient. The console contains electronic components and circuitry including memory and one or more processors. The memory includes executable instructions configured to cause the console to instantiate one or more ultrasound-imaging processes (“ultrasound-imaging process[es]”) for ultrasound imaging with the ultrasound probe. The ultrasound-imaging process(es) include an image-generating process for generating one or more ultrasound images (“ultrasound image[s]”) from the echoed ultrasound signals for at least a target area of the patient for establishing the vascular access.

The AR device includes a mechanical support supporting electronic components and circuitry including memory and one or more processors. The memory includes executable instructions configured to cause the AR device to instantiate one or more vascular access-facilitating processes (“vascular access-facilitating process[es]”) for facilitating establishment of the vascular access with a nonmagnetic needle. The vascular access-facilitating process(es) includes a detection process, a registration process, an anchoring process, and a needle-guiding process. The detection process is for detecting one or more registration marks (“registration mark [s]”) on or about the patient from image data captured by one or more patient-facing cameras of the AR device. The registration process is for registering the registration mark(s), the AR device thereby establishing its location and orientation relative to at least the target area of the patient for establishing the vascular access. The anchoring process is for anchoring the ultrasound image(s) of the target area of the patient on or about the patient as viewed through a see-through display screen of the AR device coupled to the mechanical support, the ultrasound image(s) anchored on or about the patient relative to either an instant or previous location and orientation of the ultrasound probe provided by the image data. The needle-guiding process is for the establishing of the vascular access with the needle, the needle-guiding process providing an instant virtual needle trajectory (“VNT”) of the needle as viewed through the display screen of the AR device to indicate to a clinician whether the needle is properly oriented with respect to a target vessel of the ultrasound image(s) for the establishing of the vascular access.

In some embodiments, the registration mark(s) are selected from body parts and surface features.

In some embodiments, the body parts are selected from one or more limbs, one or more digits, one or more joints, a head, and a neck.

In some embodiments, the surface features are selected from any veins, moles, warts, scars, wrinkles, dimples, pigment changes, freckles, birthmarks, and tattoos.

In some embodiments, the registration mark(s) are selected from dots, lines, shapes, and patterns drawn on the patient by the clinician.

In some embodiments, the registration mark(s) are selected from unmarked patches, marked patches including dots, lines, shapes, or patterns drawn thereon by the clinician, and printed patches including dots, lines, shapes, or patterns printed thereon.

In some embodiments, the unmarked patches, marked patches, or the printed patches are shaped with detectable features for at least the detection process.

In some embodiments, the ultrasound image(s) are anchored on or about the patient relative to the instant location and orientation of the ultrasound probe provided by the image data. Such anchoring of the ultrasound image(s) maintains spatial attention of the clinician in the target area during the establishing of the vascular access instead of dividing the spatial attention of the clinician between the target area and a display of the console.

In some embodiments, the ultrasound image(s) are anchored on or about the patient relative to the previous location and orientation of the ultrasound probe provided by the image data. Such anchoring of the ultrasound images allows the clinician to set aside the ultrasound probe and use two hands during the establishing of the vascular access.

In some embodiments, the needle-guiding process utilizes the location and orientation of the AR device relative to the target area of the patient established in the registration process as well as a depth of the target vessel determined from the ultrasound image(s) in a vessel depth-determining process to at least visually indicate to the clinician via the instant VNT whether the needle is properly oriented with respect to the target vessel of the ultrasound image(s) for the establishing of the vascular access.

Also disclosed herein is a method of establishing vascular access with a needle-guiding system. The method includes emitting source ultrasound signals into a patient and receiving echoed ultrasound signals from the patient by way of an ultrasound sensor array disposed in a probe head of an ultrasound probe. The method also includes running ultrasound-imaging process(es) for ultrasound imaging with the ultrasound probe upon one or more processors of a console executing executable instructions stored in memory of the console. The ultrasound-imaging process(es) include an image-generating process for generating ultrasound image(s) from the echoed ultrasound signals for at least a target area of the patient for establishing the vascular access. The method also includes running vascular access-facilitating process(es) for facilitating establishment of the vascular access with a nonmagnetic needle upon one or more processors of a wearable AR device executing executable instructions stored in memory of the AR device. The running of the vascular access-facilitating process(es) includes detecting with a detection process registration mark(s) on or about the patient from image data captured by one or more patient-facing cameras of the AR device. The running of the vascular access-facilitating process(es) also includes registering the registration mark(s) with a registration process, the AR device thereby establishing its location and orientation relative to at least the target area of the patient for establishing the vascular access. The running of the vascular access-facilitating process(es) also includes anchoring with an anchoring process the ultrasound image(s) of the target area of the patient on or about the patient as viewed through a see-through display screen of the AR device, the ultrasound image(s) anchored on or about the patient relative to either an instant or previous location and orientation of the ultrasound probe provided by the image data. The running of the vascular access-facilitating process(es) also includes providing with a needle-guiding process an instant VNT of the needle as viewed through the display screen of the AR device, thereby indicating to a clinician whether the needle is properly oriented with respect to a target vessel of the ultrasound image(s) for the establishing of the vascular access.

In some embodiments, the registration mark(s) are selected from body parts and surface features.

In some embodiments, the body parts are selected from one or more limbs, one or more digits, one or more joints, a head, and a neck.

In some embodiments, the surface features are selected from any veins, moles, warts, scars, wrinkles, dimples, pigment changes, freckles, birthmarks, and tattoos.

In some embodiments, the registration mark(s) are selected from dots, lines, shapes, and patterns drawn on the patient by the clinician.

In some embodiments, the registration mark(s) are selected from unmarked patches, marked patches including dots, lines, shapes, or patterns drawn thereon by the clinician, and printed patches including dots, lines, shapes, or patterns printed thereon.

In some embodiments, the unmarked patches, marked patches, or the printed patches are shaped with detectable features for at least the detection process.

In some embodiments, the anchoring of the ultrasound image(s) on or about the patient results in the ultrasound image(s) anchored on or about the patient relative to the instant location and orientation of the ultrasound probe provided by the image data. Such anchoring facilitates maintaining spatial attention of the clinician in the target area during the establishing of the vascular access instead of dividing the spatial attention of the clinician between the target area and a display of the console.

In some embodiments, the anchoring of the ultrasound image(s) on or about the patient results in the ultrasound image(s) anchored on or about the patient relative to the previous location and orientation of the ultrasound probe provided by the image data. Such anchoring allows the clinician to set aside the ultrasound probe and use two hands during the establishing of the vascular access.

In some embodiments, the needle-guiding process utilizes the location and orientation of the AR device relative to the target area of the patient established in the registration process as well as a depth of the target vessel determined from the ultrasound image(s) in a vessel depth-determining process to at least visually indicate to the clinician via the instant VNT whether the needle is properly oriented with respect to the target vessel of the ultrasound image(s) for the establishing of the vascular access.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a needle-guiding system including an ultrasound system and a wearable AR device for establishing vascular access in accordance with some embodiments.

FIG. 2 illustrates a block diagram of the ultrasound system of the needle-guiding system in accordance with some embodiments.

FIG. 3 illustrates a block diagram of the AR device of the needle-guiding system in accordance with some embodiments.

FIG. 4 illustrates body parts and surface features of a patient useful as registration marks for the AR device in accordance with some embodiments.

FIG. 5 illustrates an ultrasound image anchored on or about a patient relative to an instant location and orientation of the ultrasound probe together with an instant VNT of a needle that would be successful for establishing vascular access in accordance with some embodiments.

FIG. 6 illustrates the ultrasound image showing a successful establishment of vascular access with the needle in accordance with some embodiments.

FIG. 7 illustrates an ultrasound image anchored on or about a patient relative to a previous location and orientation of the ultrasound probe together with an instant VNT of a needle that would be successful for establishing vascular access in accordance with some embodiments.

FIG. 8 illustrates the ultrasound image showing a successful establishment of vascular access with the needle in accordance with some embodiments.

FIG. 9 illustrates an ultrasound image anchored on or about a patient relative to a previous location and orientation of the ultrasound probe together with virtual anatomical structures and an instant VNT of a needle that would be unsuccessful in establishing vascular access in accordance with some embodiments.

FIG. 10 illustrates the ultrasound image together with the virtual anatomical structures and an instant VNT of the needle that would be successful in establishing vascular access in accordance with some embodiments.

FIG. 11 illustrates the ultrasound image showing a successful establishment of vascular access with the needle in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “alternative reality,” alternative reality includes virtual reality, augmented reality, and mixed reality unless context suggests otherwise. “Virtual reality” includes virtual content in a virtual setting, which setting can be a fantasy or real-world simulation. “Augmented reality” and “mixed reality” include virtual content in a real-world setting. Augmented reality includes the virtual content in the real-world setting, but the virtual content is not necessarily anchored in the real-world setting. For example, the virtual content can be information overlying the real-world setting. The information can change as the real-world setting changes due to time or environmental conditions in the real-world setting, or the information can change as a result of an experiencer of the augmented reality moving through the real-world setting, but the information remains overlying the real-world setting. Mixed reality includes the virtual content anchored in every dimension of the real-world setting. For example, the virtual content can be a virtual object anchored in the real-world setting. The virtual object can change as the real-world setting changes due to time or environmental conditions in the real-world setting, or the virtual object can change to accommodate the perspective of an experiencer of the mixed reality as the experiencer moves through the real-world setting. The virtual object can also change in accordance with any interactions with the experiencer or another real-world or virtual agent. Unless the virtual object is moved to another location in the real-world setting by the experiencer of the mixed reality, or some other real-world or virtual agent, the virtual object remains anchored in the real-world setting. Mixed reality does not exclude the foregoing information overlying the real-world setting described in reference to augmented reality.

Unless defined otherwise, all technical and scientific terms used herein have the same meaning as commonly understood by those of ordinary skill in the art.

As set forth above, vascular access by way of, for example, placement of a catheter into a peripheral blood vessel provides an effective means for withdrawing blood, transfusing blood, delivering medications, or providing nutrition to a patient over a period of days, weeks, or even months. Such vascular access is initially established by a percutaneous puncture with a needle, and a variety of ultrasound systems having magnetic needle-tracking technology exist to facilitate first-stick success when establishing vascular access. While the foregoing ultrasound systems greatly improve first-stick success, a clinician must divide his or her spatial attention between two different spatial regions when establishing vascular access. Indeed, the clinician must divide his or her spatial attention between 1) a target area of a patient for the vascular access, where an ultrasound probe is used for ultrasound imaging, and 2) a display rendering corresponding ultrasound images of the target area. Having to divide spatial attention between the target area and the display can be difficult when simultaneously ultrasound imaging and establishing vascular access to a peripheral blood vessel with a needle. Notably, such difficulty can be pronounced with less experienced clinicians and older clinicians having reduced lens flexibility in their eyes. Further, the foregoing ultrasound systems also require the clinician to divide use of his or her hands between two different tasks, specifically, manipulating the ultrasound probe for the ultrasound imaging and manipulating the needle for establishing vascular access. What is needed are needle-guiding systems that allow clinicians to maintain their spatial attention in the target area while establishing vascular access, as well as provide the option of using both hands for establishing vascular access.

Disclosed herein are needle-guiding systems and methods that address the foregoing needs. Notably, the needle-guiding systems and methods do not utilize magnetic needle-tracking technology, so neither a magnetic needle nor magnetic sensors are needed. Thus, there are no issues with magnetic interference in the needle-guiding systems and methods disclosed herein.

### Needle-Guiding Systems

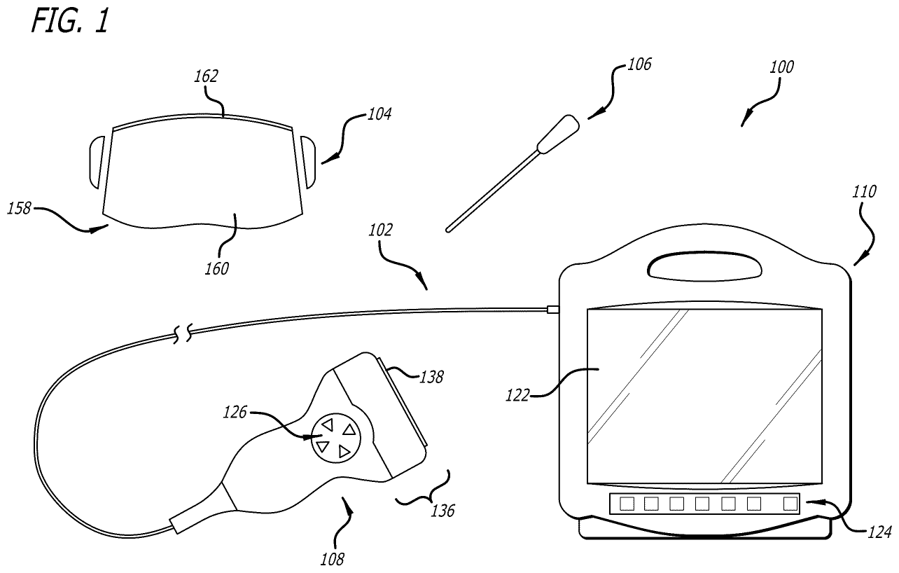

FIG. 1 illustrates a needle-guiding system 100 including an ultrasound system 102 and a wearable AR device 104 for establishing vascular access in accordance with some embodiments.

As shown, the needle-guiding system 100 can include the ultrasound system 102, the wearable AR device 104, and optionally, a nonmagnetic needle 106 for establishing vascular access. That is, the needle 106 can be considered part of the needle-guiding system 100 or separate element therefrom. The ultrasound system 102, in turn, can include an ultrasound probe 108 and a console 110. However, it should be understood that the ultrasound system 102 is not limited to the ultrasound probe 108 and the console 110 set forth below. Indeed, the ultrasound system 102 can include the ultrasound probe 108 modified to operate with a mobile device such as a smartphone instead of the console 110. Alternatively, the ultrasound system 102 can include the ultrasound probe 108 modified to operate with the AR device 104, itself. In such embodiments, the ultrasound probe 108 can be modified to include a wireless communications module (not shown) to operably communicate with the mobile device or the AR device 104 wirelessly as opposed to over a probe interface like that of the console 110. Further, the mobile device or the AR device 104 can include an application installed thereon for ultrasound imaging as opposed to an embedded system like that of the console 110. As such, a combination of hardware and software for the ultrasound system 102 can vary provided ultrasound-imaging functionality of the ultrasound system 102 remains in the combination of hardware and software for effectuating needle guidance with the needle-guiding system 100.

### Ultrasound System

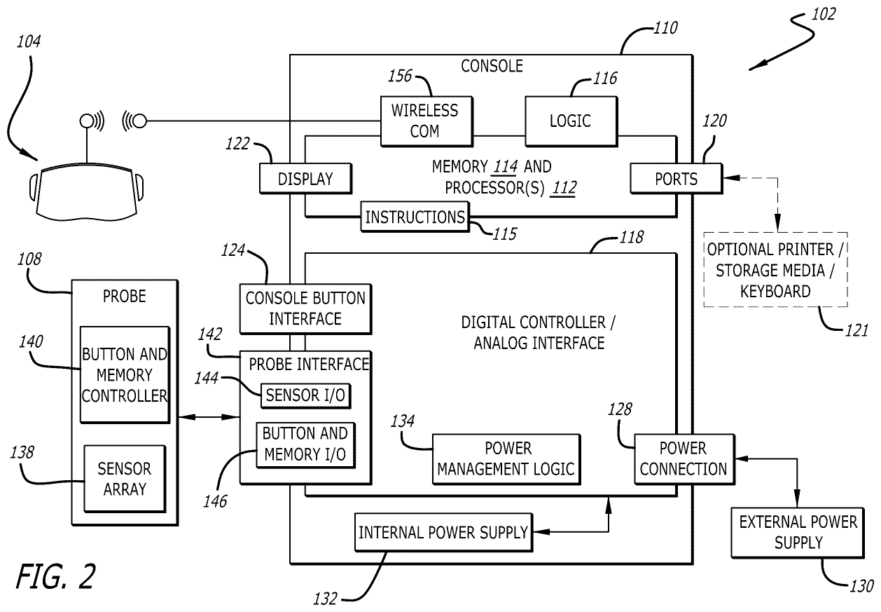

FIG. 2 illustrates a block diagram of the ultrasound system 102 of the needle-guiding system 100 in accordance with some embodiments.

As shown, the console 110 can include electronic circuitry and components including one or more processors (“processor [s]”) 112, memory 114, and logic 116 for instantiating or running one or more processes, as set forth in more detail below. The processor(s) 112 and the memory 114 (e.g., non-volatile memory such as electrically erasable, programmable, read-only memory [“EEPROM”]) of the console 110 can be configured for controlling various functions of the needle-guiding system 100 such as ultrasound imaging and virtualization of one or more physical anatomical structures (“physical anatomical structure [s]”), as set forth below. Further, the console 110 can include a digital controller or analog interface 118 in operable communication with the processor(s) 112, the memory 114, and any one or more other components of the needle-guiding or ultrasound system 100 or 102 operably connected to the console 110, for example, the AR device 104 or the ultrasound probe 108, to govern operation between them.

The console 110 can also include ports 120 for operably connecting additional or optional components 121 of the needle-guiding or ultrasound system 100 or 102 including peripheral devices such as a standalone monitor, storage media, a printer, or the like. The ports 120 can be universal serial bus (“USB”) ports; however, ports other than USB ports as well as combinations of the foregoing ports can be incorporated into the console 110. For example, the ports 102 can include a USB-C port, a DisplayPort (“DP”) port, and a high-definition multimedia interface (“HDMI”) port, or some combination thereof for operably connecting the display 122 if separate from the console 110 such as that in the foregoing standalone monitor.

The console 110 can also include a display 122 such as a liquid crystal display (“LCD”) integrated into the console 110 to display information to a clinician before, during, or after establishing vascular access with the needle-guiding system 100. For example, the display 122 can be used to display the ultrasound image(s) 150 of the target area 152 of the patient attained by the ultrasound probe 108. Alternatively, the display 122 can be separate from the console 110 such as in the standalone monitor set forth above instead of integrated into the console 110. However, it should be understood that any such display is different than that of the AR device 104. Notably, the console 110 can also include a console button interface 124. In combination with control buttons 126 on the ultrasound probe 108, the console button interface 124 of the console 110 can be used by the clinician to immediately call up a desired mode of the needle-guiding or ultrasound system 100 or 102 on the display 122 for use by the clinician in establishing vascular access.

Lastly, the console 110 can also include a power connection 128 to enable an operable connection of the console 110 to an external power supply 130. The console 110 can also include an internal power supply 132 (e.g., disposable or rechargeable battery) together with the external power supply 130 or exclusive of the external power supply 130. Power management logic 134 with the digital controller or analog interface 118 of the console 110 can regulate power use and distribution within the console 110 as well as at least some of the additional or optional components of the needle-guiding or ultrasound system 100 or 102 when such components are operably connected to the console 110.

The ultrasound probe 108 can include a probe head 136 configured to be placed against skin in the target area 152 of the patient and held in place, rotated, translated, or some combination thereof over the skin of the patient. The probe head 136 of the ultrasound probe 108 can, in turn, include an ultrasound sensor array 138 disposed in the probe head 136, which ultrasound sensor array 138 can include piezoelectric transducers or capacitive micromachined ultrasound transducers (“CMUTs”). The ultrasound probe 108 or the probe head 136 thereof can thusly be placed against the skin of the patient and held in place or slid over the skin of the patient while the ultrasound sensor array 138 emits source ultrasound signals into the patient and receives echoed ultrasound signals from the patient.

The ultrasound probe 108 can further include a button-and-memory controller 140 for governing operation of the ultrasound probe 108 and the control buttons 126 thereof. The button-and-memory controller 140 can include non-volatile memory such as EEPROM. When the ultrasound probe 108 is operably connected to the console 110, the button-and-memory controller 140 can be in operable communication with a probe interface 142 of the console 110; however, as set forth above, the ultrasound probe 108 can include the wireless communications module (not shown) to operably communicate with a mobile device, the AR device 104, or even the console 110 and its wireless communications module 156 wirelessly as opposed to over the probe interface 142. That said, the probe interface 142 of the console 110 can include an ultrasound-sensor input-output component 144 for operably communicating with the ultrasound sensor array 138 of the ultrasound probe 108 and a button-and-memory input-output component 146 for operably communicating with the button-and-memory controller 140 of the ultrasound probe 108.

Again, the processor(s) 112 and the memory 114 of the console 110 can be configured for instantiating or running one or more processes, which, in turn, can control various functions of the needle-guiding system 100 such as ultrasound imaging and virtualization of the physical anatomical structure(s). Indeed, the memory 114 can include executable instructions 115 stored thereon configured to cause the console 110 to instantiate or run ultrasound-imaging process(es) for the ultrasound imaging with the ultrasound probe 108. Such ultrasound-imaging process(es) can include an image-generating process, as set forth in more detail below. In addition, the instructions 115 stored on the memory 114 can be configured to cause the console 110 to instantiate or run one or more virtualization processes (“virtualization process[es]”) for the virtualization of the physical anatomical structure(s) imaged by the ultrasound system 102 as one or more virtual anatomical structures (“virtual anatomical structure [s]”) 148. Such virtualization process(es) can include a frame-capturing process, a frame-stitching process, a segmenting process, a modeling process, or some combination thereof, each of which is set forth in more detail below.

Beginning with ultrasound-imaging process(es), the ultrasound-imaging process(es) can include an image-generating process for generating ultrasound image(s) 150 from the echoed ultrasound signals echoed from at least a target area 152 of a patient including a potential target vessel for establishing vascular access. Such an image-generating process can utilize image-generating logic of the logic 116 to determine tissue depths from echo lengths of time for the echoed ultrasound signals as well as assign greyscale values in accordance with intensities of the echoed ultrasound signals, which, in turn, correspond to pixels in the ultrasound image(s) 150. Relatedly, the executable instructions 115 stored on the memory 114 can be configured to cause the console 110 to instantiate a vessel depth-determining process for determining vessel depths for the potential and non-target vessels set forth herein. Such a vessel depth-determining process can utilize vessel depth-determining logic of the logic 116 to determine the vessel depths from the echo lengths of time for the echoed ultrasound signals or the ultrasound image(s) 150, optionally, together with machine-learning or computer-vision vessel recognition in accordance with vessel characteristics predefined in the vessel depth-determining logic such as size or shape of the potential and non-target vessels.

Continuing with the virtualization process(es), the frame-capturing process can utilize frame-capturing logic of the logic 116 for capturing ultrasound-imaging frames (i.e., frame-by-frame ultrasound images) in a frame buffer of the memory 114 of the console 110 during the image-generating process. The capturing of the ultrasound-imaging frames can be event-based capturing in accordance with the frame-capturing logic such as when a potential target vessel is detected via target-detection logic of the logic 116 in one or more of the ultrasound-imaging frames.

The frame-stitching process can utilize frame-stitching logic of the logic 116 for stitching the ultrasound-imaging frames together in stitched ultrasound-imaging frames either during the image-generating process or sometime thereafter. The stitching of the ultrasound-imaging frames can be aligned in accordance with the frame-stitching logic as made necessary by the probe head 136 of the ultrasound probe 108 being held in place, rotated, translated, or some combination thereof over the skin of the patient during the image-generating process.

The segmenting process can utilize segmenting logic of the logic 116 for segmenting the ultrasound-imaging frames or the stitched ultrasound-imaging frames into ultrasound-image segments corresponding to the physical anatomical structure(s) of the patient. The segmenting of the ultrasound-imaging frames or the stitched ultrasound-imaging frames can be target-based segmenting in accordance with the segmenting logic such as when a potential target vessel is detected via the target-detection logic in one or more of the ultrasound-imaging frames.

The modeling process can utilize modeling logic of the logic 116 for modeling the virtual anatomical structure(s) 148 from the ultrasound-image segments such that the virtual anatomical structure(s) 148 correspond to the physical anatomical structure(s) in three-dimensional (“3D”) space. Notably, the ultrasound-image segments can be incomplete with holes or include minor intrasegment misalignments for which holes and misalignments the modeling logic can correct in the virtual anatomical structure(s) 148 by filling in the holes and align the misalignments. That said, the ultrasound-imaging frames captured during the frame-capturing process can be highly redundant, thereby mostly obviating the filling of such holes and the aligning over of such misalignments.

Notwithstanding the foregoing, it should be understood that the virtualization process(es) are not limited to the virtualization of the potential target vessels. Indeed, any physical anatomical structure imaged by the ultrasound system 102 including any non-target vessel or structure can be virtualized as a virtual anatomical structure. For example, the capturing of the ultrasound-imaging frames by the frame-capturing process need not be event-based capturing as set forth above. Instead, the capturing of the ultrasound-imaging frames can be continuous so as to capture ultrasound-imaging frames including both the potential target vessels and non-target vessels or structures. Notably, the target-detection logic can still be utilized in the frame-capturing process to at least differentiate between captured ultrasound-imaging frames including the potential target vessels and those including only the non-target vessels or structures, which can reduce processing time for the virtualization of the potential target vessels. In addition, the segmenting of the ultrasound-imaging frames or the stitched ultrasound-imaging frames into the ultrasound-image segments by the segmenting process need not be limited to target-based segmenting as set forth above. Instead, the segmenting of the ultrasound-imaging frames or the stitched ultrasound-imaging frames can be inclusive so as to segment the ultrasound-imaging frames or the stitched ultrasound-imaging frames into ultrasound-image segments corresponding to both the potential target vessels and the non-target vessels or structures. Notably, the target-detection logic can also still be utilized in the segmenting process to at least preferentially segment the ultrasound-imaging frames or the stitched ultrasound-imaging frames into the ultrasound-image segments corresponding to the potential target vessels, which can further reduce the processing time for the virtualization of the potential target vessels.

The modeling of the virtual anatomical structure(s) 148 can also be inclusive so as to model both the potential target vessels and the non-target vessels or structures from the ultrasound-image segments. Notably, the target-detection logic can also be utilized in the modeling process to at least preferentially model the potential target vessels from the ultrasound-image segments, which can even further reduce processing time for the virtualization of the potential target vessels. Virtualization of both the potential target vessels and the non-target vessels or structures facilitates establishing vascular access without adverse events such as unintended arterial punctures during venipunctures. Indeed, virtualization of both the potential target vessels and the non-target vessels or structures shows the clinician the potential target vessels as they exist among the non-target vessels or structures from which the clinician can select a target vessel 154 from the potential target vessels having a relatively low risk of puncturing a neighboring non-target vessel or structure should the target vessel be missed with the needle 106. Lastly, each of the ultrasound-imaging process(es) and the virtualization process(es) can independently include a sending process configured to respectively send the ultrasound image(s) 150 and the virtual anatomical structure(s) 148 to the AR device 104 for display on or about the patient by way of a wireless communications module 156 of the console 110.

### AR Device

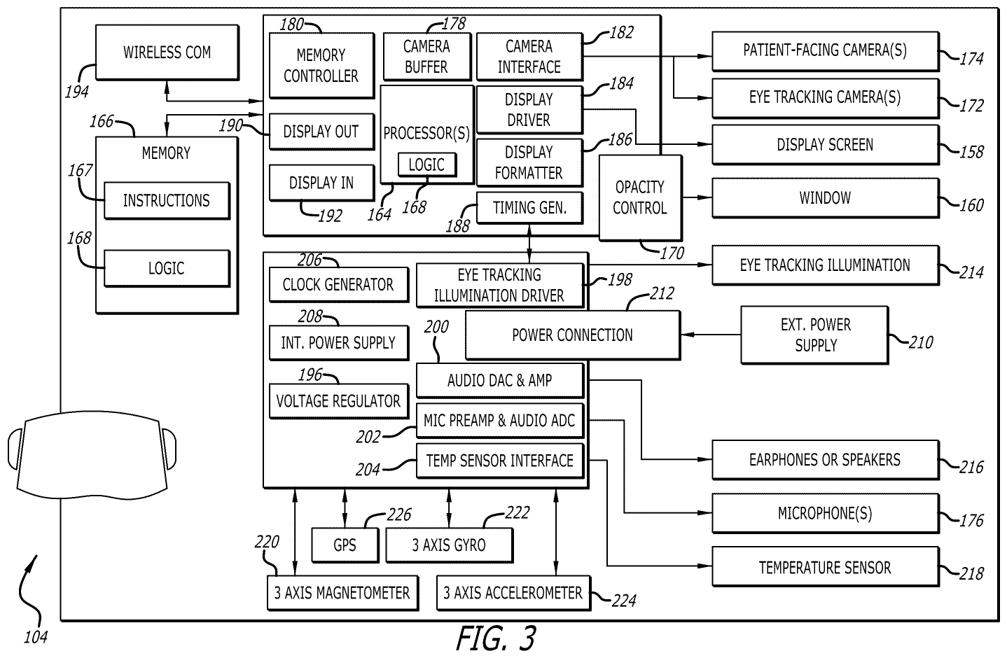

FIG. 3 illustrates a block diagram of the AR device 104 of the needle-guiding system 100 in accordance with some embodiments.

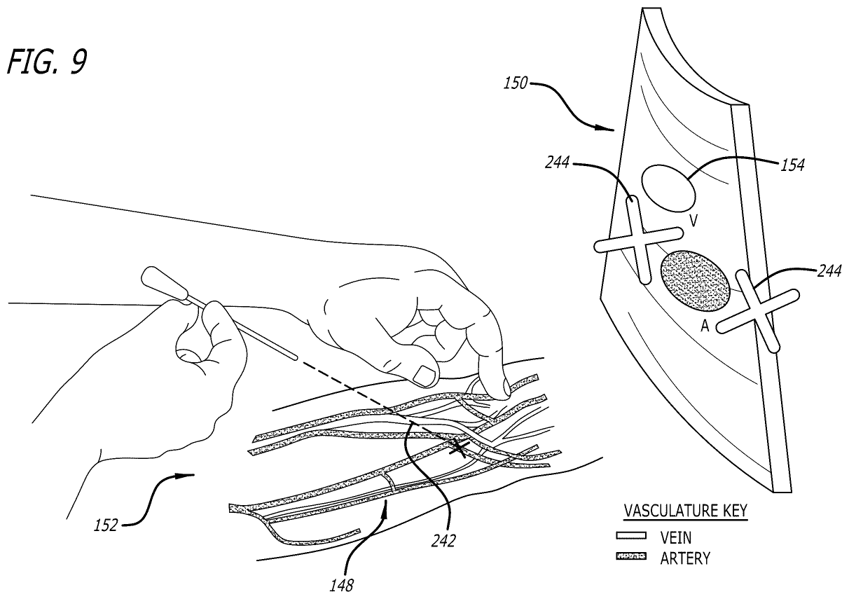

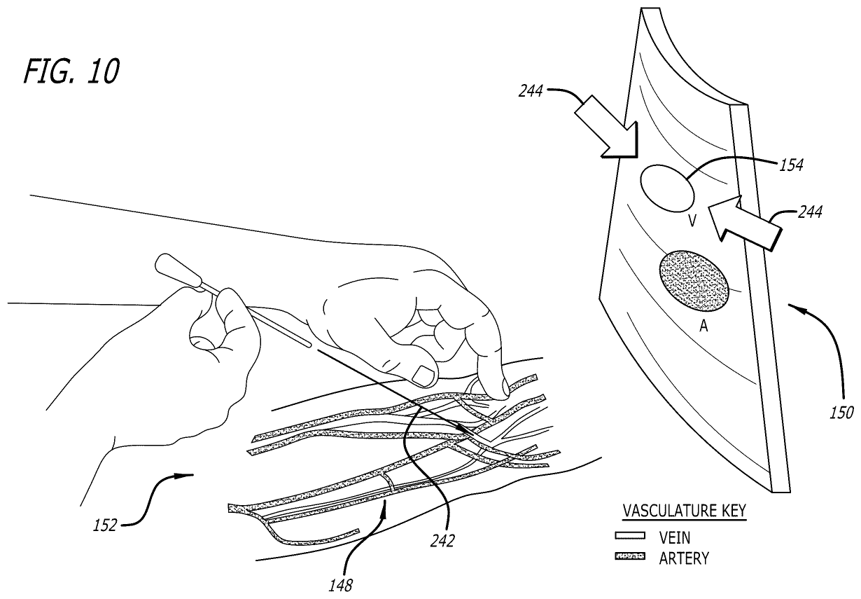

As shown, the AR device 104 can include a suitably configured display 158 and a window 160 thereover coupled to a mechanical support such as a frame 162 supporting electronic circuitry and components including processor(s) 164, memory 166 (e.g., dynamic random-access memory [“DRAM”]), and logic 168 for instantiating or running one or more processes, as set forth in more detail below. The display 158 can be configured such that a wearer of the AR device 104 such as the clinician can see an environment (e.g., examination room, operating room, etc.) including the patient through the display 158 in accordance with an opacity of the window 160, which opacity can be adjustable with an opacity control 170 to change a degree of opacity of the window 160. The display 158 can be configured to display the virtual anatomical structure(s) 148 over the environment such as on or about the patient. (See, for example, FIGS. 9 and 10, wherein the virtual anatomical structure(s) 148 correspond to the vasculature in a limb of the patient.) In displaying the virtual anatomical structure(s) 148 over the environment, the AR device 104 can be configured to three-dimensionally anchor the virtual anatomical structure(s) 148 to the environment such as to the patient over which the virtual anatomical structure(s) 148 are displayed, which allows the wearer of the AR device 104 to see a true representation of the patient's anatomy for establishing vascular access. Anchoring the virtual anatomical structure(s) 148 to the environment or to the patient over which the virtual anatomical structure(s) 148 are displayed is characteristic of mixed reality.

The AR device 104 can further include a perceptual user interface (“PUI”) configured to enable the wearer of the AR device 104 to interact with the AR device 104 without a physical input device such as keyboard or mouse. Instead of a physical input device, the PUI can have input devices including, but not limited to, one or more wearer-facing eye-tracking cameras (“eye-tracking camera [s]”) 172, one or more patient-facing cameras (“patient-facing camera [s]”) 174, one or more microphones (“microphone [s]”) 176, or a combination thereof. At least one advantage of the PUI and the input devices thereof is that the clinician does not have to reach outside a sterile field to execute a command of the AR device 104.

As to the eye-tracking camera(s) 172, the eye-tracking camera(s) 172 can be coupled to the frame 162 and configured to capture eye movements of the wearer in a camera buffer 178 or the memory 166. The processor(s) 164 of the AR device 104 can be configured to process the eye movements with eye-movement logic of the logic 168 to identify a focus of the wearer for selecting the virtual anatomical structure(s) 148 corresponding to the focus of the wearer. For example, the focus of the wearer can be used by the PUI to select a particular target vessel of the potential target vessels such as the target vessel 154 for enhancing it by way of highlighting the target vessel 154 or increasing the contrast between the target vessel 154 and the potential target vessels, the non-target vessels or structure, or the environment. In another example, the focus of the wearer can be used by the PUI to select a particular target vessel of the potential target vessels such as the target vessel 154 for performing one or more other operations of the PUI such as zooming in on the target vessel 154 among a remainder of the potential target vessels and the non-target vessels or structures, so the clinician can ensure there is a relatively low risk of puncturing a neighboring non-target vessel or structure should the target vessel 154 be missed with the needle 106.

The patient-facing camera(s) 174 can be coupled to the frame 162 and configured to capture gestures of the wearer in the camera buffer 178 or the memory 166. The processor(s) 164 of the AR device 104 can be configured to process the gestures with gesture-command logic of the logic 168 to identify gesture-based commands issued by the wearer for execution thereof by the AR device 104. Further, the patient-facing camera(s) 174 can be configured to capture image data in the camera buffer 178 or the memory 166 corresponding to the registration mark(s) 228 on or about the patient for the detection process of the vascular access-facilitating process(es) set forth below.

The microphone(s) 176 can be coupled to the frame 162 configured to capture audio of the wearer in the memory 166. The processor(s) 164 of the AR device 104 can be configured to process the audio with audio-command logic of the logic 168 to identify audio-based commands issued by the wearer for execution thereof by the AR device 104.

In addition to the processor(s) 164, the memory 166, and the logic 168, the electronic circuitry and components can include a memory controller 180 in communication with the memory 166, a camera interface 182, the camera buffer 178, a display driver 184, a display formatter 186, a timing generator 188, a display-out interface 190, and a display-in interface 192. Such components can be in communication with each other through the processor(s) 164, dedicated lines of one or more buses, or some combination thereof.

The camera interface 182 can be configured to provide an interface to the eye-tracking camera(s) 172 and the patient-facing camera(s) 172 and 174, as well as store respective images received from the cameras 172 and 174 in the camera buffer 178 or the memory 166. Each camera of the eye-tracking camera(s) 172 can be an infrared (“IR”) camera or a position-sensitive detector (“PSD”) configured to track eye-glint positions by way of IR reflections or eye glint-position data, respectively.

The display driver 184 can be configured to drive the display 158. The display formatter 186 can be configured to provide display-formatting information for the virtual anatomical structure(s) 148 to the processor(s) 112 of the console 110 for formatting the virtual anatomical structure(s) 148 for display on the display 158 over the environment such as on or about the patient. The timing generator 188 can be configured to provide timing data for the AR device 104. The display-out interface 190 can include a buffer for providing images from the eye-tracking camera(s) 172 or the patient-facing camera(s) 174 to the processor(s) 112 of the console 110. The display-in interface 192 can include a buffer for receiving images such as the virtual anatomical structure(s) 148 to be displayed on the display 158. The display-out and display-in interfaces 190 and 192 can be configured to communicate with the console 110 by way of a wireless communications module 194.

Further, the electronic circuitry and components can include a voltage regulator 196, an eye-tracking illumination driver 198, an audio digital-to-analog converter (“DAC”) and amplifier 200, a microphone preamplifier and audio analog-to-digital converter (“ADC”) 202, a temperature-sensor interface 204, and a clock generator 206. The voltage regulator 196 can be configured to receive power from an internal power supply 208 (e.g., a battery) or an external power supply 210 through a power connection 212. The voltage regulator 196 can be configured to provide the received power to the electronic circuitry of the AR device 104. The eye-tracking illumination driver 198 can be configured to control an eye-tracking illumination unit 214 by way of a drive current or voltage to operate about a predetermined wavelength or within a predetermined wavelength range. The audio DAC and amplifier 200 can be configured to provide audio data to earphones or speakers 216. The microphone preamplifier and audio ADC 202 can be configured to provide an interface for the microphone(s) 176. The temperature sensor interface 204 can be configured as an interface for a temperature sensor 218. In addition, the AR device 104 can include orientation sensors including a three-axis magnetometer 220, a three-axis gyroscope 222, and a three-axis accelerometer 224 configured to provide orientation-sensor data for determining an orientation of the AR device 104 at any given time. The AR device 104 can even further include a global-positioning system (“GPS”) receiver 226 configured to receive GPS data (e.g., time and position information for one or more GPS satellites) for determining a location of the AR device 104 at any given time.

Again, the processor(s) 164 and the memory 166 of the AR device 104 can be configured for instantiating or running one or more processes, which, in turn, can control various functions of the needle-guiding system 100 such as facilitating vascular access. Indeed, the memory 166 can include executable instructions 167 stored thereon configured to cause the AR device 104 to instantiate or run vascular access-facilitating process(es) for facilitating establishment of the vascular access with the needle 106. Such vascular access-facilitating process(es) can include a detection process, a registration process, an anchoring process, a needle-guiding process, or some combination thereof, as set forth in more detail below.

The detection process can utilize registration mark-detecting logic of the logic 168 for detecting registration mark(s) 228 on or about the patient from the image data captured by the patient-facing camera(s) 174 of the AR device 104. The detecting of the registration mark(s) 228 can include machine-learning or computer-vision feature recognition in accordance with body-part or surface-feature characteristics predefined in the registration mark-detecting logic such as size or shape of one or more body parts (“body part [s]”); size, shape, color, or texture of one or more surface characteristics (“surface characteristic [s]”); or distance between the body part(s), the surface characteristic(s), or any other registration marks set forth herein. Indeed, it should be understood the registration mark(s) 228 and, thus, the detecting of the registration mark(s) 228 is not limited to the foregoing body part(s) and surface feature(s).

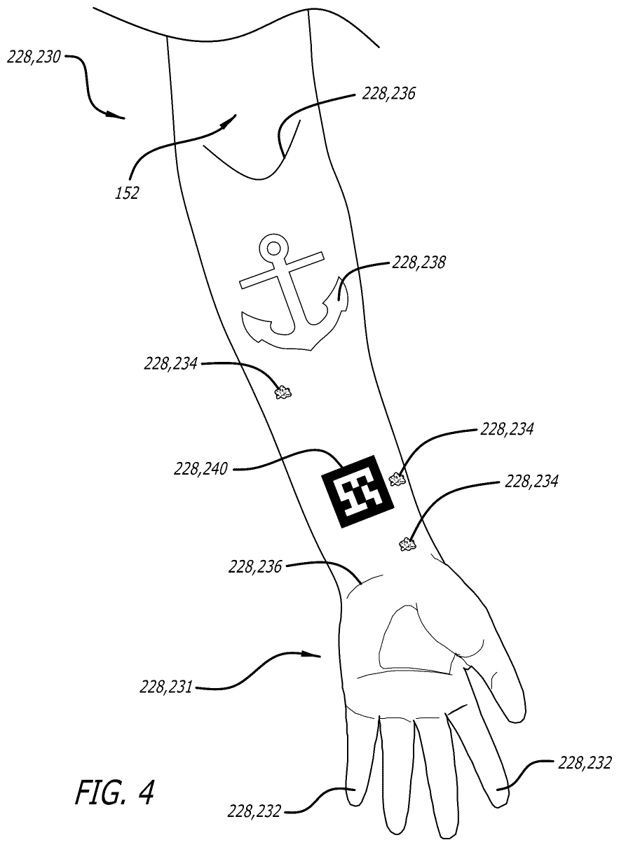

FIG. 4 illustrates the registration mark(s) 228 including the body part(s) and surface feature(s) of the patient in accordance with some embodiments.

As to the registration mark(s) 228, the registration mark(s) 228 can be selected from the foregoing body part(s) and surface feature(s) as well as articles of manufacture and clinician markings intended to function as registration marks. In an example, the registration mark(s) 228 can include the body part(s) selected from one or more limbs including arms 230 or legs or portions thereof such as hands 231 and feet; one or more digits including fingers 232 or toes; one or more joints including shoulders, elbows, wrists, knees, or ankles; a head, in its entirety, or cars, eyes, lips, or a nose thereof; and a neck, in its entirety, or a laryngeal prominence thereof. In another example, the registration mark(s) 228 can include the surface feature(s) selected from veins including protruding surface veins, varicose veins, spider veins; moles 234; warts; scars; wrinkles 236; dimples; pigment changes; freckles; birthmarks; and tattoos 238. In yet another example, the registration mark(s) 228 can include the articles of manufacture selected from unmarked or blank patches and printed patches 240 including dots, lines, shapes, patterns, or some combination thereof printed on the printed patches 240. In yet another example, the registration mark(s) 228 can include the clinician markings selected from dots, lines, shapes, and patterns drawn on the patient, any of the foregoing patches, or somewhere in the environment by the clinician. Notably, the unmarked patches, marked patches marked on by the clinician, or the printed patches 240 can be shaped with or otherwise include detectable features for at least the detection process. Such features can include lobes or other extensions of the patches, angled or rounded corners of the patches, angles between any two sides of the patches, edge-based features along one or more sides of the patches such as perforation used for separating the patches from a larger sheet thereof, or the like.

The registration process can utilize registration mark-registering logic of the logic 168 for registering the registration mark(s) 228 detected in the detection process in the memory 166 of the AR device 104. The registering of the registration mark(s) 228 can include registering the registration mark(s) 228 in the memory 166 against the orientation-sensor data from the orientation sensors as well as the GPS data from the GPS receiver 226, the AR device 104 thereby establishing its location and orientation relative to the registration mark(s) 228 on or about the patient and, therefore, any target area 152 of the patient for establishing vascular access.

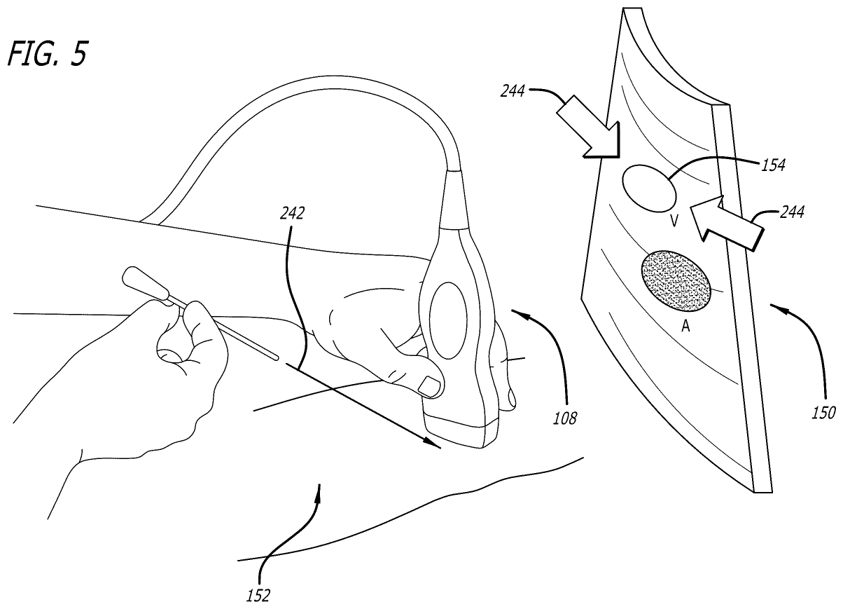

The anchoring process can utilize anchoring logic of the logic 168 for anchoring the ultrasound image(s) 150 of the target area 152 of the patient on or about the patient in an AR user interface (“ARUI”) for viewing through the display 158 of the AR device 104. The anchoring of the ultrasound image(s) 150 on or about the patient in the ARUI can be proximate or relative to either an instant or previous location and orientation of the ultrasound probe 108, which locations and orientations of the ultrasound probe 108 can be provided by way of the image data captured by the patient-facing camera(s) 174 of the AR device 104. Indeed, should the ultrasound probe 108 include any registration mark(s) 228 applied thereto or integrated therewith, the detection and registration processes can further detect and register such registration mark(s) 228 for the anchoring of the ultrasound image(s) 150 on or about the patient in the ARUI relative to either the instant or previous location and orientation of the ultrasound probe 108 as shown in FIGS. 5-11 and described in further detail below. Notably, anchoring of the ultrasound image(s) 150 on or about the patient in the ARUI maintains spatial attention of the clinician in the target area 152 of the patient while establishing vascular access with the needle 106 instead of dividing the spatial attention of the clinician between the target area 152 and the display 122 of the console 110.

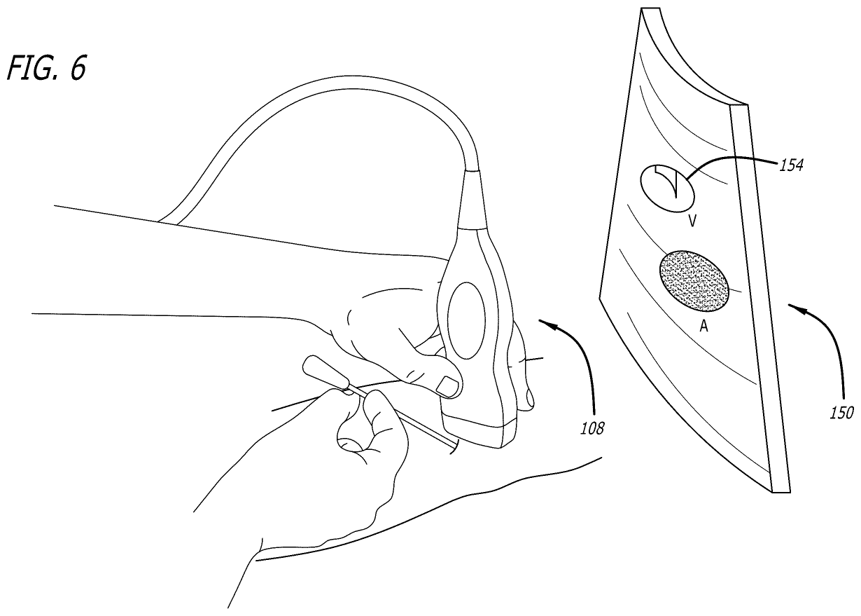

The needle-guiding process can utilize needle-guiding logic of the logic 168 for guiding the needle 106 for establishing vascular access with the needle 106. The guiding of the needle 106 can include providing a virtual element such as an instant VNT 242 of the needle 106 for viewing in the ARUI through the display 158 of the AR device 104, optionally, together with the ultrasound image(s) 150 and any additional virtual elements provided therewith, to indicate to the clinician whether the needle 106 is properly oriented with respect to the target vessel 154 of the patient such as that identified in the ultrasound image(s) 150 for establishing vascular access. Indeed, the needle-guiding process can utilize the location and orientation of the AR device 104 relative to the target area 152 of the patient established in the registration process of the AR device 104 as well as the vessel depths of the potential and non-target vessels determined in the vessel depth-determining process of the console 110 to at least visually indicate to the clinician via the VNT 242 whether the needle 106 is properly oriented with respect to the target vessel 154 of the ultrasound image(s) 150 for establishing vascular access. While the guiding of the needle 106 by the VNT 242 together with the ultrasound image(s) 150 and any additional virtual elements is optional, the ultrasound image(s) 150 instantaneously and advantageously allow the clinician to witness, notably, without diverting his or her spatial attention, whether he or she is successful in establishing vascular access as shown in FIGS. 6, 8, and 11. That, and one or more additional virtual elements 244 (“additional virtual element[s]”) can be advantageously visually coordinated with the VNT 242 as shown in FIGS. 5, 7, 9, and 10 to increase the probability the clinician is successful in establishing vascular access as shown in FIGS. 6, 8, and 11.

The VNT 242 can visually indicate to the clinician whether the needle 106 is properly oriented with respect to the target vessel 154 of the patient for establishing vascular access thereto. For example, the VNT 242 can visually indicate whether the needle 106 is properly oriented with respect to the target vessel 154 by line type such as a broken or dashed VNT (see FIG. 9) for an improper orientation of the needle 106 and an unbroken or solid VNT (see FIGS. 5, 7, and 10) for a proper orientation; line end such as an ‘X’ at a distal end of the VNT 242 (see FIG. 9) for an improper orientation of the needle 106 and an arrow at the distal end of the VNT 242 (see FIGS. 5, 7, and 10) for a proper orientation; line color such as red for an improper orientation of the needle 106 and green for a proper orientation; visual effects such as a glowing, breathing, or pulsing effect for an improper orientation of the needle 106 and no such effect for a proper orientation; or a combination thereof. Further, as set forth above, the additional virtual element(s) 244, if present, can be visually coordinated with the VNT 242 to indicate to the clinician whether the needle 106 is properly oriented with respect to the target vessel 154 of the patient. For example, the additional virtual element(s) 244 can visually indicate whether the needle 106 is properly oriented with respect to the target vessel 154 by an ‘X’ over the ultrasound image(s) 150 corresponding to the ‘X’ at the distal end of the VNT 242 (see FIG. 9) for an improper orientation of the needle 106 and an arrow over the ultrasound image(s) 150 corresponding to the arrow at the distal end of the VNT 242 (see FIGS. 5, 7, and 10) for a proper orientation. Any such additional virtual element can be over the ultrasound image(s) 150 at a depth corresponding to following the VNT 242 into a plane of an ultrasound image of the ultrasound image(s) 150. Notably, should the VNT 242 be unsuitable for viewing by any clinician while establishing vascular accesses with the needle 106, the VNT 242 can be disabled via the PUI from showing in the ARUI in favor of the additional virtual element(s) 244 indicating to the clinician whether the needle 106 is properly oriented for establishing vascular access to the target vessel 154 of the patient such as that of the ultrasound image(s) 150.

In an example, FIG. 5 illustrates the ultrasound image(s) 150 anchored on or about the patient relative to an instant location and orientation of the ultrasound probe 108 together with the instant VNT 242 of the needle 106 and the additional virtual element(s) 244 that would be successful for establishing vascular access to the target vessel 154 of the patient. Notably, anchoring of the ultrasound image(s) 150 on or about the patient relative to the instant location and orientation of the ultrasound probe 108 can be referred to as a synchronous access mode of the needle-guiding system 100, which, without a securing means for securing the ultrasound probe 108 in its location and orientation, generally involves two hands divided between two different tasks while establishing vascular access to the target vessel 154 with the needle 106, those tasks being manipulating the ultrasound probe 108 for the ultrasound imaging and manipulating the needle 106 for establishing vascular access. However, such anchoring of the ultrasound image(s) 150 advantageously allows the clinician to maintain spatial attention in the target area 152 while establishing vascular access instead of dividing his or her spatial attention between the target area 152 and the display 122 of the console 110, thereby increasing the probability the clinician is successful in establishing vascular access as shown in FIG. 6.

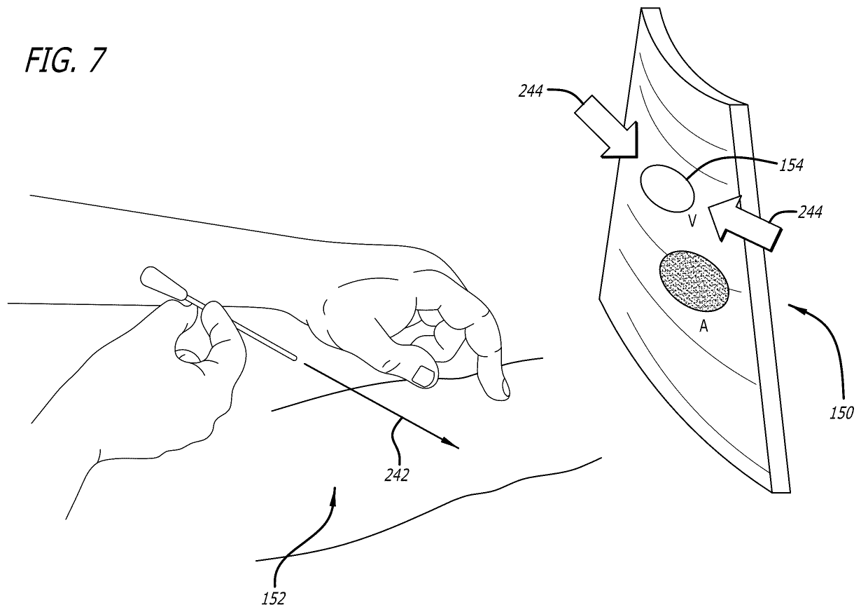

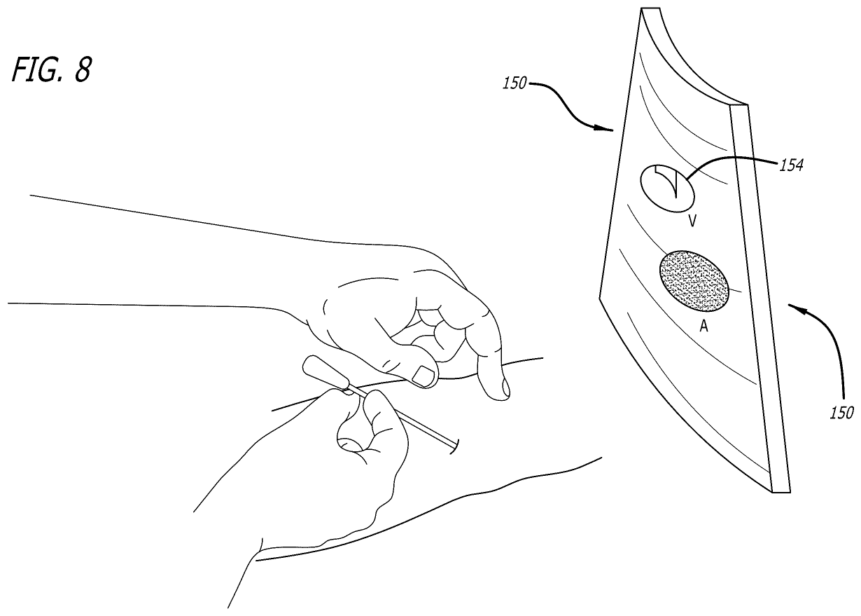

In another example, FIG. 7 illustrates the ultrasound image(s) 150 anchored on or about the patient relative to a previous location and orientation of the ultrasound probe 108, as provided by the image data captured by the patient-facing camera(s) 174 of the AR device 104, together with the instant VNT 242 of the needle 106 and the additional virtual element(s) 244 that would be successful for establishing vascular access to the target vessel 154 of the patient. Notably, anchoring of the ultrasound image(s) 150 on or about the patient relative to the previous location and orientation of the ultrasound probe 108 can be referred to as an asynchronous access mode of the needle-guiding system 100, which advantageously provides the clinician the option to set aside the ultrasound probe 108 and use two hands in the single task of manipulating and, optionally, steadying the needle 106 while establishing vascular access to the target vessel 154 with the needle 106. That said, should both hands not be needed to manipulate or steady the needle 106, a free hand can be utilized to manipulate the patient, the skin of the patient about an insertion site of the target area 152, or the like. Indeed, the free hand can even be utilized to obtain the ultrasound probe 108 for additional ultrasound imaging upon momentarily switching back to the synchronous access mode via the PUI. Like that set forth above, anchoring of the ultrasound image(s) 150 also advantageously allows the clinician to maintain spatial attention in the target area 152 while establishing vascular access instead of dividing his or her spatial attention between the target area 152 and the display 122 of the console 110, thereby increasing the probability the clinician is successful in establishing vascular access as shown in FIG. 8.

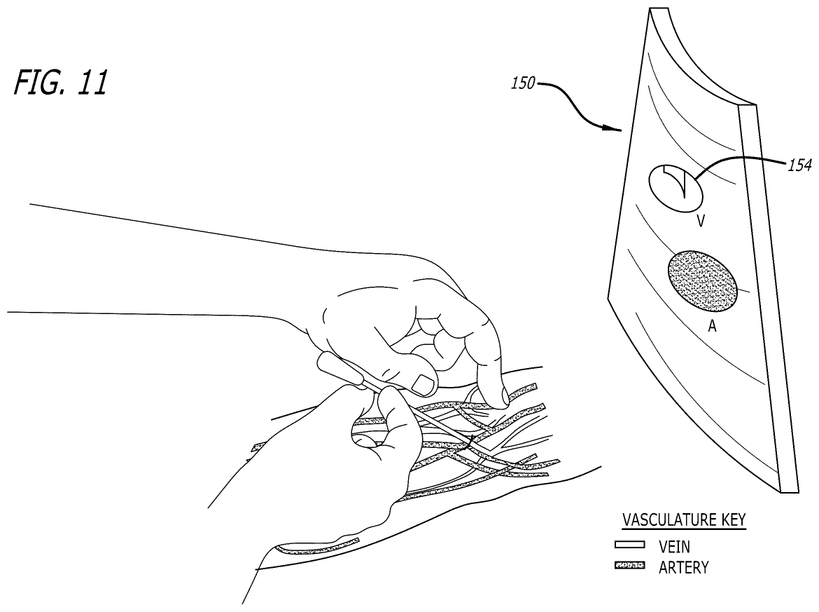

In another example, FIG. 9 illustrates the ultrasound image(s) 150 anchored on or about the patient relative to a previous location and orientation of the ultrasound probe 108, as provided by the image data captured by the patient-facing camera(s) 174 of the AR device 104, together with the instant VNT 242 of the needle 106 and the additional virtual element(s) 244 that would be unsuccessful for establishing vascular access to the target vessel 154 of the patient. In contrast, FIG. 10 illustrates the ultrasound image(s) 150 anchored on or about the patient relative to the previous location and orientation of the ultrasound probe 108 together with the instant VNT 242 of the needle 106 and the additional virtual element(s) 244 that would be successful for establishing vascular access to the target vessel 154 of the patient. Indeed, as set forth above, the ‘X’ at the distal end of the VNT 242 and each ‘X’ shown for the additional virtual element(s) 244 in FIG. 9 visually indicate the needle 106 is improperly oriented with respect to the target vessel 154 and, therefore, would be unsuccessful for establishing vascular access to the target vessel 154 of the patient. But the arrow at the distal end of the VNT 242 and each arrow shown for the additional virtual element(s) 244 in FIG. 10 visually indicate the needle 106 is properly oriented with respect to the target vessel 154 and, therefore, would be successful for establishing vascular access to the target vessel 154 of the patient. Further, in addition to features and advantages set forth above with respect to FIGS. 7 an 8 for the asynchronous access mode of the needle-guiding system 100, FIGS. 9 and 10 illustrate the virtual anatomical structure(s) 148 as vasculature in a limb of the patient together with the instant VNT 242 of the needle 106 and the additional virtual element(s) 244. Such virtual anatomical structure(s) 148 allow the clinician to see a true representation of the vasculature in the limb of the patient to increase the probability the clinician is successful in establishing vascular access as shown in FIG. 11.

### Methods

Methods include methods of the needle-guiding system 100, itself, as well as methods of using the needle-guiding system 100 to establish vascular access with the needle 106. Indeed, a method of establishing vascular access with the needle-guiding system 100 is set forth below. Any method using the needle-guiding system 100 to establish vascular access can be gleaned from at least description set forth above.

A method of establishing vascular access with the needle-guiding system 100 can include running the ultrasound-imaging process(es) for ultrasound imaging with the ultrasound probe 108 upon the processor(s) 112 of the console 110 executing the executable instructions 115 stored in the memory 114 of the console 110. Indeed, the ultrasound-imaging process(es) can include emitting source ultrasound signals into the patient and receiving echoed ultrasound signals from the patient by way of the ultrasound sensor array 138 disposed in the probe head 136 of the ultrasound probe 108 such as when holding or sliding the probe head 136 over the skin of the patient. The ultrasound-imaging process(es) can also include the image-generating process for generating the ultrasound image(s) 150 from the echoed ultrasound signals for at least the target area 152 of the patient for establishing vascular access.

The method of establishing vascular access with the needle-guiding system 100 can also include running the vascular access-facilitating process(es) for facilitating establishment of the vascular access with the nonmagnetic needle 106 upon the processor(s) 164 of the wearable AR device 104 executing the executable instructions 167 stored in the memory 166 of the AR device 104. As set forth above, the running of the vascular access-facilitating process(es) can include detecting with the detection process the registration mark(s) 228 on or about the patient from the image data captured by the patient-facing camera(s) 174 of the AR device 104. The running of the vascular access-facilitating process(es) can also include registering the registration mark(s) 228 with the registration process, the AR device 104 thereby establishing its location and orientation relative to at least the target area 152 of the patient for establishing vascular access. The running of the vascular access-facilitating process(es) can also include anchoring with the anchoring process the ultrasound image(s) 150 of the target area 152 of the patient on or about the patient as viewed through the see-through display 158 of the AR device 104, the ultrasound image(s) 150 anchored on or about the patient relative to either an instant or previous location and orientation of the ultrasound probe 108 provided by the image data. Lastly, the running of the vascular access-facilitating process(es) an also include providing with the needle-guiding process the instant VNT 242 of the needle 106 as viewed through the display 158 of the AR device 104, thereby indicating to the clinician whether the needle 106 is properly oriented with respect to the target vessel 154 of the ultrasound image(s) 150 for establishing vascular access thereto.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A needle-guiding system for establishing vascular access, comprising:
an ultrasound probe including an ultrasound sensor array disposed in a probe head of the ultrasound probe, the ultrasound sensor array configured to emit source ultrasound signals into a patient and receive echoed ultrasound signals from the patient when holding or sliding the probe head over skin of the patient;
a console containing electronic components and circuitry including memory and one or more processors, the memory including executable instructions configured to cause the console to instantiate one or more ultrasound-imaging processes (“ultrasound-imaging process[es]”) for ultrasound imaging with the ultrasound probe, and the ultrasound-imaging process(es) including an image-generating process for generating one or more ultrasound images (“ultrasound image[s]”) from the echoed ultrasound signals for at least a target area of the patient for establishing the vascular access; and
a wearable alternative-reality (“AR”) device including a mechanical support supporting electronic components and circuitry including memory and one or more processors, the memory including executable instructions configured to cause the AR device to instantiate one or more vascular access-facilitating processes (“vascular access-facilitating process[es]”) for facilitating establishment of the vascular access with a nonmagnetic needle, and the vascular access-facilitating process(es) including:
a detection process for detecting one or more registration marks (“registration mark [s]”) on or about the patient from image data captured by one or more patient-facing cameras of the AR device;
a registration process for registering the registration mark(s), the AR device thereby establishing its location and orientation relative to at least the target area of the patient for establishing the vascular access;
an anchoring process for anchoring the ultrasound image(s) of the target area of the patient on or about the patient as viewed through a see-through display screen of the AR device coupled to the mechanical support, the ultrasound image(s) anchored on or about the patient relative to either an instant or previous location and orientation of the ultrasound probe provided by the image data; and
a needle-guiding process for the establishing of the vascular access with the needle, the needle-guiding process providing an instant virtual needle trajectory (“VNT”) of the needle as viewed through the display screen of the AR device to indicate to a clinician whether the needle is properly oriented with respect to a target vessel of the ultrasound image(s) for the establishing of the vascular access.

2. The needle-guiding system of claim 1, wherein the registration mark(s) are selected from body parts and surface features.

3. The needle-guiding system of claim 2, wherein the body parts are selected from one or more limbs, one or more digits, one or more joints, a head, and a neck.

4. The needle-guiding system of claim 2, wherein the surface features are selected from any veins, moles, warts, scars, wrinkles, dimples, pigment changes, freckles, birthmarks, and tattoos.

5. The needle-guiding system of claim 1, wherein the registration mark(s) are selected from dots, lines, shapes, and patterns drawn on the patient by the clinician.

6. The needle-guiding system of claim 1, wherein the registration mark(s) are selected from unmarked patches, marked patches including dots, lines, shapes, or patterns drawn thereon by the clinician, and printed patches including dots, lines, shapes, or patterns printed thereon.

7. The needle-guiding system of claim 6, wherein the unmarked patches, marked patches, or the printed patches are shaped with detectable features for at least the detection process.

8. The needle-guiding system of claim 1, wherein the ultrasound image(s) are anchored on or about the patient relative to the instant location and orientation of the ultrasound probe provided by the image data, thereby maintaining spatial attention of the clinician in the target area during the establishing of the vascular access instead of dividing the spatial attention of the clinician between the target area and a display screen of the console.

9. The needle-guiding system of claim 1, wherein the ultrasound image(s) are anchored on or about the patient relative to the previous location and orientation of the ultrasound probe provided by the image data, thereby allowing the clinician to set aside the ultrasound probe and use two hands during the establishing of the vascular access.

10. The needle-guiding system of claim 1, wherein the needle-guiding process utilizes the location and orientation of the AR device relative to the target area of the patient established in the registration process as well as a depth of the target vessel determined from the ultrasound image(s) in a vessel depth-determining process to at least visually indicate to the clinician via the instant VNT whether the needle is properly oriented with respect to the target vessel of the ultrasound image(s) for the establishing of the vascular access.

11. A method of establishing vascular access with a needle-guiding system, comprising:
emitting source ultrasound signals into a patient and receiving echoed ultrasound signals from the patient by way of an ultrasound sensor array disposed in a probe head of an ultrasound probe;
running one or more ultrasound-imaging processes (“ultrasound-imaging process[es]”) for ultrasound imaging with the ultrasound probe upon one or more processors of a console executing executable instructions stored in memory of the console, the ultrasound-imaging process(es) including an image-generating process for generating one or more ultrasound images (“ultrasound image[s]”) from the echoed ultrasound signals for at least a target area of the patient for establishing the vascular access; and
running one or more vascular access-facilitating processes (“vascular access-facilitating process[es]”) for facilitating establishment of the vascular access with a nonmagnetic needle upon one or more processors of a wearable alternative-reality (“AR”) device executing executable instructions stored in memory of the AR device, the running of the vascular access-facilitating process(es) including:
detecting with a detection process one or more registration marks (“registration mark [s]”) on or about the patient from image data captured by one or more patient-facing cameras of the AR device;
registering the registration mark(s) with a registration process, the AR device thereby establishing its location and orientation relative to at least the target area of the patient for establishing the vascular access;
anchoring with an anchoring process the ultrasound image(s) of the target area of the patient on or about the patient as viewed through a see-through display screen of the AR device, the ultrasound image(s) anchored on or about the patient relative to either an instant or previous location and orientation of the ultrasound probe provided by the image data; and
providing with a needle-guiding process an instant virtual needle trajectory (“VNT”) of the needle as viewed through the display screen of the AR device, thereby indicating to a clinician whether the needle is properly oriented with respect to a target vessel of the ultrasound image(s) for the establishing of the vascular access.

12. The method of claim 11, wherein the registration mark(s) are selected from body parts and surface features.

13. The method of claim 12, wherein the body parts are selected from one or more limbs, one or more digits, one or more joints, a head, and a neck.

14. The method of claim 12, wherein the surface features are selected from any veins, moles, warts, scars, wrinkles, dimples, pigment changes, freckles, birthmarks, and tattoos.

15. The method of claim 11, wherein the registration mark(s) are selected from dots, lines, shapes, and patterns drawn on the patient by the clinician.

16. The method of claim 11, wherein the registration mark(s) are selected from unmarked patches, marked patches including dots, lines, shapes, or patterns drawn thereon by the clinician, and printed patches including dots, lines, shapes, or patterns printed thereon.

17. The method of claim 16, wherein the unmarked patches, marked patches, or the printed patches are shaped with detectable features for at least the detection process.

18. The method of claim 11, wherein the anchoring of the ultrasound image(s) on or about the patient results in the ultrasound image(s) anchored on or about the patient relative to the instant location and orientation of the ultrasound probe provided by the image data, which facilitates maintaining spatial attention of the clinician in the target area during the establishing of the vascular access instead of dividing the spatial attention of the clinician between the target area and a display screen of the console.

19. The method of claim 11, wherein the anchoring of the ultrasound image(s) on or about the patient results in the ultrasound image(s) anchored on or about the patient relative to the previous location and orientation of the ultrasound probe provided by the image data, which allows the clinician to set aside the ultrasound probe and use two hands during the establishing of the vascular access.

20. The method of claim 11, wherein the needle-guiding process utilizes the location and orientation of the AR device relative to the target area of the patient established in the registration process as well as a depth of the target vessel determined from the ultrasound image(s) in a vessel depth-determining process to at least visually indicate to the clinician via the instant VNT whether the needle is properly oriented with respect to the target vessel of the ultrasound image(s) for the establishing of the vascular access.
